(19) United States
(12) Patent Application Publication
(10) Pub. No.: US 2022/0378306 A1
(43) Pub. Date: Dec. 1, 2022

(54) APPARATUS AND METHOD FOR ESTIMATING BIO-INFORMATION

(71) Applicants: SAMSUNG ELECTRONICS CO., LTD., Suwon-si (KR); IUCF-HYU (INDUSTRY-UNIVERSITY COOPERATION FOUNDATION HANYANG UNIVERSITY), Seoul (KR)

(72) Inventors: Sang Kon BAE, Seongnam-si (KR); Joon-Hyuk CHANG, Seoul (KR); Jin Woo CHOI, Suwon-si (KR); Jehyun KYUNG, Seoul (KR); Youn Ho KIM, Hwaseong-si (KR); Inmo YEON, Seoul (KR)

(73) Assignees: SAMSUNG ELECTRONICS CO., LTD., Suwon-si (KR); IUCF-HYU (INDUSTRY-UNIVERSITY COOPERATION FOUNDATION HANYANG UNIVERSITY), Seoul (KR)

(21) Appl. No.: 17/404,238

(22) Filed: Aug. 17, 2021

(30) Foreign Application Priority Data
May 27, 2021 (KR) 10-2021-0068286

(51) Int. Cl. A61B 5/021; A61B 5/00; A61B 5/02; A61B 5/16; G06N 3/04; G16H 10/60; G16H 50/30

(52) U.S. Cl. CPC A61B 5/02108; A61B 5/6843; A61B 5/7264; A61B 5/7239; A61B 5/02007; A61B 5/165; A61B 5/442; G06N 3/0454; G16H 10/60; G16H 50/30; A61B 2562/0247

(57) ABSTRACT

An apparatus for estimating bio-information is provided. According to an embodiment of the present disclosure, the apparatus for estimating bio-information includes: a pulse wave sensor having a plurality of channels to measure a plurality of pulse wave signals from an object; a force sensor configured to obtain a force signal by measuring an external force exerted onto the pulse wave sensor; and a processor configured to: obtain a first feature for each channel by inputting the plurality of pulse wave signals for each channel and the force signal, into a first neural network model; obtain a weight for each channel by inputting the first feature to a second neural network model; obtain a second feature by applying the weight to the first feature for each channel by using the second neural network model; and obtain bio-information by inputting the second feature to a third neural network model.

### CROSS-REFERENCE TO RELATED APPLICATION(S)

This application claims priority from Korean Patent Application No. 10-2021-0068286, filed on May 27, 2021, in the Korean Intellectual Property Office, the disclosure of which is incorporated herein by reference in its entirety.

### BACKGROUND

### 1. Field

Apparatuses and methods consistent with example embodiments relate to non-invasively estimating bio-information, and more particularly to estimating bio-information by applying a deep learning-based estimation model.

### 2. Description of the Related Art

Generally, methods of non-invasively measuring blood pressure without causing pain to a human body include a method to measure blood pressure by measuring a cuff-based pressure and a method to estimate blood pressure by measuring pulse waves without the use of a cuff. A Korotkoff-sound method is one of cuff-based blood pressure measurement methods, in which a pressure in a cuff wound around an upper arm is increased and blood pressure is measured by monitoring the sound generated in the blood vessel through a stethoscope while decreasing the pressure. Another cuff-based blood pressure measurement method is an oscillometric method using an automated machine, in which a cuff is wound around an upper arm, a pressure in the cuff is increased, a pressure in the cuff is continuously measured while the cuff pressure is gradually decreased, and blood pressure is measured based on a point where a change in a pressure signal is large. Cuffless blood pressure measurement methods generally include a method of estimating blood pressure by calculating a Pulse Transit Time (PTT), and a Pulse Wave Analysis (PWA) method of estimating blood pressure by analyzing a pulse wave shape.

### SUMMARY

According to an aspect of an example embodiment, there is provided an apparatus for estimating bio-information, the apparatus including: a pulse wave sensor having a plurality of channels to measure a plurality of pulse wave signals from an object; a force sensor configured to obtain a force signal by measuring an external force exerted onto the pulse wave sensor; and a processor configured to: obtain a first feature for each channel by inputting the plurality of pulse wave signals for each channel and the force signal, into a first neural network model; obtain a weight for each channel by inputting the first feature to a second neural network model; obtain a second feature by applying the weight to the first feature for each channel by using the second neural network model; and obtain bio-information by inputting the second feature to a third neural network model.

The first neural network model, the second neural network model, and the third neural network model are based on at least one of a Deep Neural Network, a Convolution Neural Network (CNN), and a Recurrent Neural Network (RNN).

The first neural network model may include: three neural networks, which are executed in parallel, and into which a first input value, a second input value, and a third input value are input respectively; and a first fully connected layer configured to output the first feature for each channel by using outputs of the three neural networks as inputs.

The processor may be further configured to: generate a first order differential signal and a second order differential signal from the plurality of pulse wave signals, obtain at least one of the plurality of pulse wave signals, the first order differential signal, and the second order differential signal as the first input value; generate at least one envelope, among an envelope of the plurality of pulse wave signals, an envelope of the first order differential signal, and an envelope of the second order differential signal by using the force signal; obtain the generated at least one envelope as the second input value; and obtain the force signal as the third input value.

The second neural network model may include: an attention layer configured to generate the weight for each channel by using the first feature as an input; and a Softmax function layer configured to convert the weight for each channel into a probability value and output the probability value.

The second neural network model is configured to perform matrix multiplication of the probability value for each channel, and the first feature for each channel, and output the second feature based on results of the matrix multiplication.

The third neural network model may include: a second fully connected layer using the second feature as an input; and a third fully connected layer configured to output the bio-information by using an output of the second fully connected layer as an input.

The weight for each channel based on the first feature is a first weight. The apparatus may include: a fourth neural network model configured to generate a second weight for each channel based on a third feature for each channel, which is extracted based on at least one of the force signal and the plurality of pulse wave signals for each channel, and output a fourth feature by applying the weight to the third feature for each channel.

The third neural network model may further include a fourth fully connected layer using the fourth feature and at least one of user characteristic information as an input, wherein an output of the fourth fully connected layer may be input into a third fully connected layer.

The user characteristic information may include at least one of a user's age, stature, and weight.

The bio-information may include one or more of blood pressure, vascular age, arterial stiffness, aortic pressure waveform, vascular compliance, stress index, fatigue level, skin age, and skin elasticity.

According to an aspect of another example embodiment, there is provided a method of estimating bio-information, the method including: by using a pulse wave sensor, acquiring a plurality of pulse wave signals for each channel from an object; by using a force sensor, acquiring a force signal applied between the object and the pulse wave sensor; obtaining a first feature for each channel by inputting the force signal and the plurality of pulse wave signals for each channel into a first neural network model; obtaining a weight for each channel by inputting the first feature into a second neural network model; obtaining a second feature by applying the weight to the first feature for each channel by using the second neural network model; and obtaining bio-information by inputting the second feature into a third neural network model.

The first neural network model, the second neural network model, and the third neural network model may use at least one of a Deep Neural Network (DNN), a Convolution Neural Network (CNN), and a Recurrent Neural Network (RNN).

The obtaining of the first feature for each channel may include: obtaining a first input value, a second input value, and a third input value for each channel; inputting the first, the second, and the third input values in parallel into three neural networks of the first neural network model; and obtaining the first feature by inputting outputs of the three neural networks into a first fully connected layer.

The first input value may include at least one of the plurality of pulse wave signals, a first order differential signal of the pulse wave signal, and a second order differential signal of the pulse wave signal. The second input value may include at least one of an envelope of the plurality of pulse wave signals, an envelope of the first order differential signal, and an envelope of the second order differential signal which are generated by using the force signal. The third input value may include the force signal.

The obtaining of the second feature may include: generating the weight for each channel by inputting the first feature into an attention layer; and converting the weight for each channel into a probability value by using a Softmax function.

The obtaining of the second feature may further include obtaining the second feature by performing matrix multiplication of the probability value for each channel and the first feature for each channel.

The obtaining the bio-information may include: inputting the second feature into a second fully connected layer; and obtaining the bio-information by inputting an output of the second fully connected layer into a third fully connected layer.

The weight for each channel based on the first feature may be a first weight, and the method may further include: generating a second weight for each channel based on a third feature for each channel, which is extracted based on at least one of the force signal and the plurality of pulse wave signals for each channel, by using a fourth neural network model; and obtaining a fourth feature by applying the second weight to the third feature for each channel.

The obtaining the bio-information may include: inputting the fourth feature and at least one of user characteristic information into a fourth fully connected layer; and outputting the bio-information by inputting an output of the fourth fully connected layer into a third fully connected layer.

### BRIEF DESCRIPTION OF THE DRAWINGS

The above and/or other aspects will be more apparent by describing certain example embodiments, with reference to the accompanying drawings, in which:

FIG. 1 is a block diagram illustrating an apparatus for estimating bio-information according to an embodiment of the present disclosure;

FIGS. 2A to 2G are diagrams explaining examples of a configuration of a processor according to the embodiment of FIG. 1;

FIG. 3A is a diagram illustrating an example of a pulse wave signal acquired by a pulse wave sensor;

FIG. 3B is a diagram illustrating an example of a force signal acquired by a force sensor;

FIG. 3C is a diagram illustrating an example of an oscillometric envelope obtained by using a pulse wave signal and a force signal;

FIG. 4 is a block diagram illustrating an apparatus for estimating bio-information according to an embodiment of the present disclosure;

FIG. 5 is a flowchart illustrating a method of estimating bio-information according to an embodiment of the present disclosure;

FIG. 6 is a flowchart illustrating an example of an operation of outputting a first feature for each channel of FIG. 5;

FIG. 7 is a flowchart illustrating an example of an operation of outputting a second feature of FIG. 5;

FIG. 8 is a flowchart illustrating a method of estimating bio-information in the case where a fourth neural network model is included, according to another embodiment of the present disclosure; and

FIGS. 9 to 11 are diagrams illustrating examples of structures of an electronic device including an apparatus for estimating bio-information.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. It will be further understood that when an element is referred to as “comprising” another element, the element is intended not to exclude one or more other elements, but to further include one or more other elements, unless explicitly described to the contrary. In the following description, terms such as “unit” and “module” indicate a unit for processing at least one function or operation and they may be implemented by using hardware, software, or a combination thereof. Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

Hereinafter, embodiments of an apparatus and method for estimating bio-information will be described in detail with reference to the accompanying drawings.

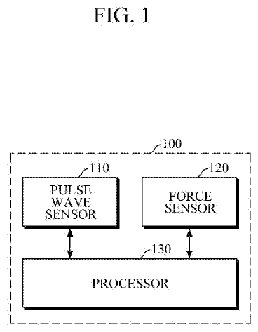

FIG. 1 is a block diagram illustrating an apparatus for estimating bio-information according to an embodiment of the present disclosure.

Referring to FIG. 1, the apparatus 100 for estimating bio-information includes a pulse wave sensor 110, a force sensor 120, and a processor 130.

The pulse wave sensor 110 may measure a pulse wave signal, including a photoplethysmography (PPG) signal, while being in contact with an object. The object may be a body part, which may come into contact with the pulse wave sensor 110, and at which pulse waves may be easily measured. For example, the object may be a finger where blood vessels are densely distributed, but the object is not limited thereto and may be an area on the wrist that is adjacent to the radial artery and an upper portion of the wrist where veins or capillaries are located, or a peripheral part of the body such as toes and the like.

The pulse wave sensor 110 may include one or more light sources emitting light onto the object, and one or more detectors disposed at a predetermined distance from the light sources and detecting light scattered or reflected from the object. The one or more light sources may emit light of different wavelengths. For example, the light sources may emit light of an infrared wavelength, a green wavelength, a blue wavelength, a red wavelength, a white wavelength, and the like. The light sources may include a light emitting diode (LED), a laser diode (LD), a phosphor, etc., but are not limited thereto. Further, the detectors may include a photodiode, a photodiode array, a complementary metal-oxide semiconductor (CMOS) image sensor, a charge-coupled device (CCD) image sensor, and the like.

The pulse wave sensor 110 may include a plurality of channels to measure a plurality of pulse wave signals at multiple points of the object. For example, the plurality of channels may have a structure including one light source and a plurality of detector arrays or CMOS image sensors disposed at a predetermined distance from the light source, or pairs of a plurality of light sources and detectors. The pulse wave sensor 110 may be implemented as a multichannel-PPG sensor that is configured to acquire a plurality of PPG signals simultaneously through a plurality of channels. The multichannel-PPG sensor may enable real-time monitoring of PPG signals. A plurality of optical signals having different wavelengths may be transmitted to and collected from a plurality of measurement points of the object, through the plurality of channels. The processor 130 may process the plurality of PPG signals that are received through the plurality of channels, independently and in parallel.

The force sensor 120 may measure a force signal when the object, being in contact with the pulse wave sensor 110, gradually increases or decreases a pressing force to induce a change in pulse wave amplitude. The force sensor 120 may be formed as a single force sensor including a strain gauge and the like, or may be formed as an array of force sensors. However, the force sensor 120 is not limited thereto, and instead of the force sensor 120, a pressure sensor, an air bladder type pressure sensor, a pressure sensor in combination with a force sensor and an area sensor, and the like may be provided.

The processor 130 may estimate bio-information based on the pulse wave signals, measured by the pulse wave sensor 110 including the plurality of channels, and the contact force measured by the force sensor 120. In this case, the bio-information may include blood pressure, vascular age, arterial stiffness, aortic pressure waveform, vascular compliance, stress index, fatigue level, skin age, skin elasticity, etc., but is not limited thereto. When a plurality of PPG signals are received through the plurality of channels, the processor 120 may process the plurality of PPG signals using a plurality of neural networks into which different sets of node weights are applied. For example, when a first PPG signal, a second PPG signal, and a third PPG signal are obtained through the plurality of channels by emitting lights of different wavelengths to the object and receiving the lights reflected from the object, the processor 120 may process the first PPG signal using a neural network constructed with a first set of node weights, may process the second PPG signal using a neural network constructed with a second set of node weights, and may process the third PPG signal using a neural network constructed with a third set of node weights, wherein the first, the second, and the third sets of node weights are different from each other.

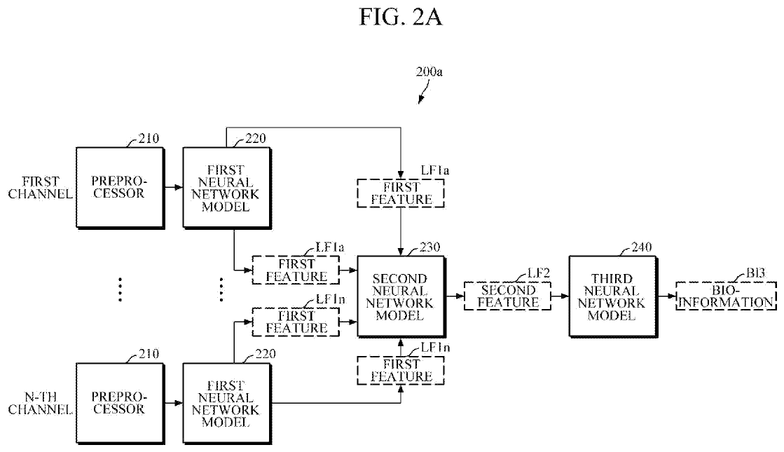

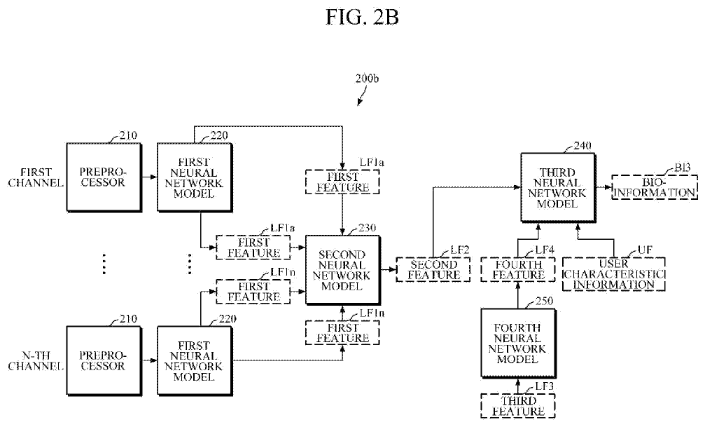

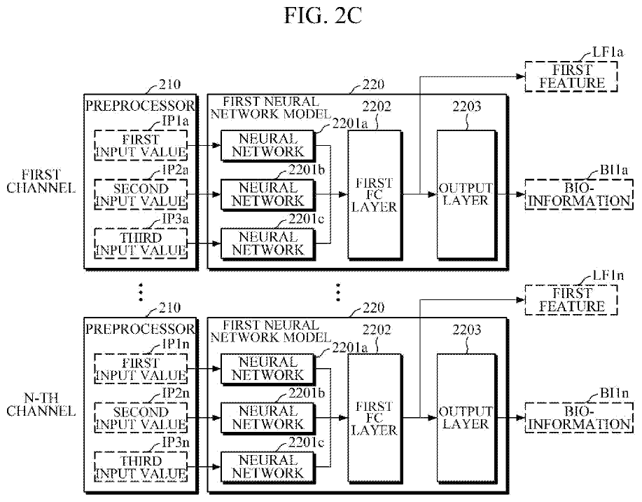

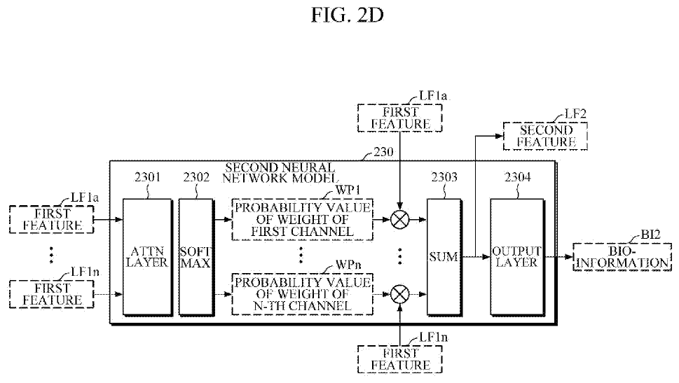

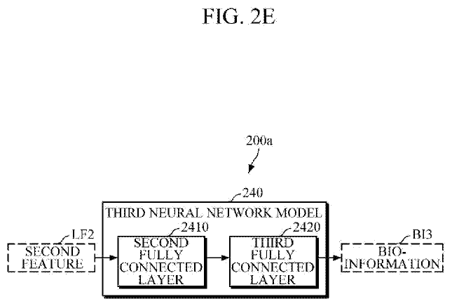

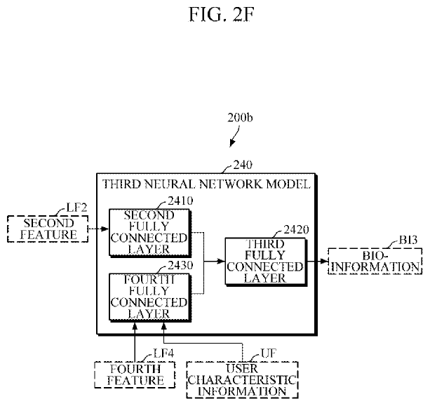

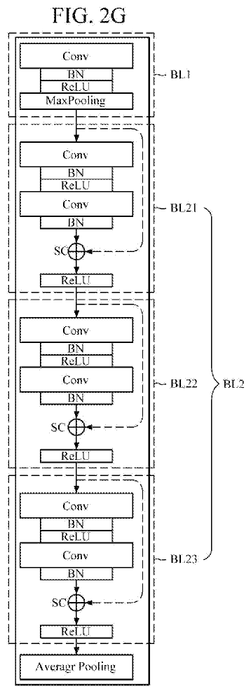

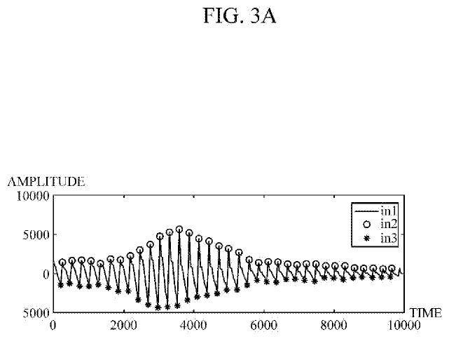

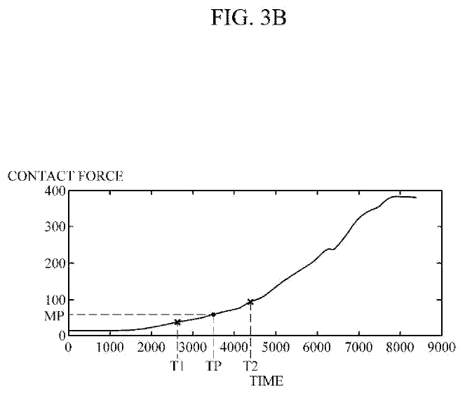

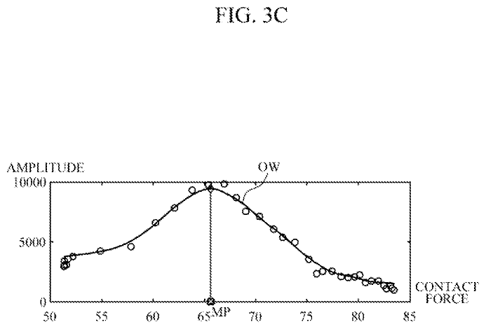

FIGS. 2A to 2G are diagrams explaining examples of a configuration of a processor according to the embodiment of FIG. 1. FIG. 3A is a diagram illustrating an example of a pulse wave signal acquired by a pulse wave sensor. FIG. 3B is a diagram illustrating an example of a force signal acquired by a force sensor. FIG. 3C is a diagram illustrating an example of an oscillometric envelope obtained by using a pulse wave signal and a force signal.

Referring to FIGS. 2A and 2G, processors 200a and 200b according to the embodiments may include a preprocessor 210, a first neural network model 220, a second neural network model 230, and a third neural network model 240. In addition, the processor 200b according to an embodiment may further include a fourth neural network model 250.

The preprocessor 210 may preprocess the pulse wave signals of the respective channels and/or the force signal by using a band pass filter and/or a low pass filter, and the like. For example, the preprocessor 210 may perform band pass filtering on the pulse wave signals, with a cut-off frequency of 1 Hz to 10 Hz.

Further, the preprocessor 210 may obtain input values to be input into neural networks by using the pulse wave signals and/or the force signal.

Referring to FIG. 2C, the preprocessor 210 may obtain, for example, first input values IP1a, . . . , and IP1n, second input values IP2a, . . . , and IP2n, and/or third input values IP3a, . . . , and IP3n for each channel by using the pulse wave signals of the respective channels.

For example, the preprocessor 210 may generate a first order differential signal and/or a second order differential signal by performing first or second order differentiation on the respective pulse wave signals, and may obtain the respective pulse wave signals, the first order differential signal and/or the second order differential signal as the first input values. In this case, signals over a predetermined time interval among the respective pulse wave signals, the first order differential signal and/or the second order differential signal may be obtained as the first input values IP1a, . . . , and IP1n. In this case, the predetermined time interval may be pre-defined based on a time point at which an amplitude of a pulse wave signal is maximum.

In addition, the preprocessor 210 may generate an envelope of a pulse wave signal, an envelope of a first order differential signal and/or an envelope of a second order differential signal by using the pulse wave signals, the first order differential signal and/or the second order differential signal, and may obtain the generated envelopes of the pulse wave signal, the first order differential signal and/or second order differential signal as the second input values IP2a, . . . , and IP2n. In this case, the predetermined time interval may be pre-defined based on a time point at which an amplitude of a pulse wave signal is maximum.

Referring to FIGS. 3A to 3C, an example of obtaining an envelope will be described below. The preprocessor 210 may extract, e.g., a peak-to-peak point of the pulse wave signal waveform by subtracting a negative (−) amplitude value in3 from a positive (+) amplitude value in2 of a waveform envelope in1 at each measurement time of the pulse wave signal. Further, the preprocessor 210 may obtain an envelope OW of the pulse wave signal by plotting the peak-to-peak amplitude at each measurement time against a contact pressure value at a corresponding time point and by performing, for example, polynomial curve fitting. Likewise, by using the first order differential signal and the force signal and/or the second order differential signal and the force signal, the preprocessor 210 may obtain an envelope of the first order differential signal and/or an envelope of the second order differential signal.

In addition, the preprocessor 210 may obtain the third input values IP3a, . . . , and IP3n by using the force signal. For example, the preprocessor 210 may determine force signals over the entire interval to be the third input values. Alternatively, as illustrated in FIG. 3B, the preprocessor 210 may determine, as the third input value, a force signal over a predetermined time interval of T1 to T2 based on a reference point TP, for example, a time interval of 5 seconds in total, with 2.5 seconds each before and after the reference point TP. In this case, the reference point TP may be a time point corresponding to a maximum amplitude point MP in an envelope of the pulse wave signal illustrated in FIG. 3C. However, the interval is not limited thereto, and an interval of force applied by the object may be pre-defined, and the interval of force may be defined differently for each user.

Referring back to FIGS. 2A and 2G, the processors 200a and 200b may include the first neural network model 220, the second neural network model 230, the third neural network model 240, and/or the fourth neural network model 250. The respective neural network models may be neural network models trained based on Deep Neural Network (DNN), Convolution Neural Network (CNN), Recurrent Neural Network (RNN), or the like.

Referring to FIG. 2C, the first neural network model 220 may include three neural networks 2201a, 2201b, and 2201c, a first fully connected layer 2202, and an output layer 2203. The respective neural networks 2201a, 2201b, and 2201c are arranged in parallel as illustrated herein, such that the first input values IP1a, . . . , and IP1n, the second input values IP2a, . . . , and IP2n, and the third input values IP3a, . . . , and IP3n, which are obtained by the preprocessor 210, may be input thereto. Further, the respective neural networks 2201a, 2201b, and 2201c may be neural networks based on a Residual Neural Network. As illustrated in FIG. 2G, the respective neural networks 2201a, 2201b, and 2201c may be composed of a first block BL1 and a second block BL2, followed by an average pooling. The first block BL1 may include convolution layer Cony, batch normalization BN, activation function ReLU, and max pooling layer MaxPooling. The second block BL2 may include one or more sub-blocks BL21, BL22, and BL23. The respective sub-blocks BL21, BL22, and BL23 may include convolution layer Cony, batch normalization BN, activation function ReLU, convolution layer Cony, batch normalization BN, skip connection SC, and activation function ReLU. In this case, the sub-blocks may be three in number, but the number is not limited thereto.

Referring back to FIG. 2C, the first fully connected layer 2202 may equalize outputs of the respective neural networks 2201a, 2201b, and 2201c, and may convert the outputs into first features LF1a, . . . , and LF1n associated with bio-information to output the features. A Sigmoid Function (not shown) may be further included after the first fully connected layer 2202. Further, the outputs of the first fully connected layer 2202 may be output as first bio-information BI1a, . . . , and BI1n through the output layer 2203.

Referring to FIG. 2D, the second neural network model 230 may include an attention layer 2301, a SoftMax function 2302, a summation function 2303, and an output layer 2304. The second neural network model 230 may be an attention network based neural network.

The attention layer 2301 may generate a weight for each channel by using, as inputs, the first features LF1a, . . . , and LF1n for each channel which correspond to the outputs of the first neural network model 220 for each channel. The SoftMax function 2302 may convert the weight for each channel into probability values WP1, . . . , and WPn, and may output the values. The term “weight” may refer to a set of weights assigned to a plurality of nodes (neuron) of a neural network that processes the first features LF1a, . . . , and LF1n.

The second neural network model 230 may perform matrix multiplication of the probability values WP1, . . . , and WPn for each channel, which are converted by the SoftMax function 2302, and the first features LF1a, . . . , and LF1n for each channel, and may sum up results of the matrix multiplication by inputting the matrix multiplication into the summation function 2303 to output a second feature LF2. Further, outputs of the summation function 2303 may be output as second bio-information BI1a, . . . , and BI1n through the output layer 2304.

For estimating bio-information, a single model, such as a DNN, CNN, etc., is generally used in methods of estimating blood pressure. Further, a single channel is generally used in methods of estimating bio-signals using a PPG signal. In this case, however, accuracy of the estimation may be reduced as the quality of signals at each position of blood vessels may vary according to the position of an object. Further, a shape of a signal slightly varies due to age, disease, medication, and the like of each individual, such that a blood pressure estimation model may be trained inaccurately. However, according to the present disclosure in which a plurality of neural network models and channels are used, by obtaining weights for each channel, which are indicative of importance, for all channels, and by performing matrix multiplication of the weights for each channel by the first features, which are outputs of the first neural network model for each channel, and by summing up results of the matrix multiplication for each channel, a new feature (e.g., second feature) is output, such that bio-information may be estimated by using the new feature obtained by comprehensively considering the features of all the channels, thereby increasing the accuracy in estimating bio-information.

Referring to FIG. 2E, the third neural network model 240 of the processor 200a according to an embodiment may include a second fully connected layer 2410 and a third fully connected layer 2420. Third bio-information BI3 may be output by the second fully connected layer 2410 using the second feature LF2 as an input, and the third fully connected layer 2420 using the output of the second fully connected layer 2410 as an input.

Referring to FIGS. 2B and 2F, the processor 200b according to another embodiment may obtain a third feature LF3 for each channel based on at least one of the pulse wave signal for each channel and a force signal.

For example, the processor 200b may obtain the third feature LF3 by extracting additional information associated with bio-information by using the pulse wave signals for each channel, the first order differential signal, the second order differential signal, and/or the force signal. For example, the third feature LF3 may include amplitude/time values at a maximum amplitude point of each signal, amplitude/time values at a local minimum point/local maximum point, amplitude/time values at an inflection point, or a total/partial area of each signal waveform, a contact force value corresponding to a maximum amplitude point, a contact force value having a predetermined ratio to the contact force value at the maximum amplitude point, or a value obtained by properly combining the information.

In addition, the processor 200b may further include a fourth neural network model 250 which generates a weight for each channel based on the third feature LF3 for each channel, and outputs a fourth feature LF4 by applying the generated weight to the third feature LF3 for each channel. The fourth neural network model has a structure similar to the second neural network model, and thus may refer to the second neural network model.

Moreover, the processor 200b may further include a fourth fully connected layer 2430 in the third neural network model 240. The fourth fully connected layer 2430 may use, as an input, at least one of the fourth feature LF4 and user characteristic information UF, and the output of the fourth fully connection layer 2430 may be input into the third fully connected layer 2420. As a result, bio-information BI3 may be estimated more accurately by using the second feature LF2, the fourth feature LF4, and the user characteristic information UF. Here, the user characteristic information UF may include at least one of a user's age, stature, and weight.

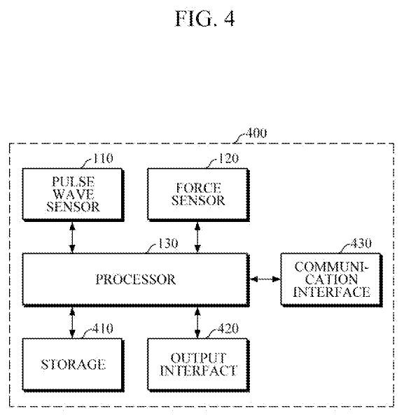

FIG. 4 is a block diagram illustrating an apparatus for estimating bio-information according to an embodiment of the present disclosure.

Referring to FIG. 4, the apparatus 400 for estimating bio-information may include the pulse wave sensor 110, the force sensor 120, the processor 130, a storage 410, an output interface 420, and a communication interface 430. The pulse wave sensor 110, the force sensor 120, and the processor 130 are described above in detail, such that the following description will be focused on non-overlapping parts.

The storage 410 may store information related to estimating bio-information. For example, the storage 410 may store data, such as the pulse wave signal, contact force, estimated bio-information value, feature vector, user characteristic information, etc., which are processed by the pulse wave sensor 110, the force sensor 120, and the processor 130. In addition, the storage 410 may include the user characteristic information, the first neural network model, the second neural network model, the third neural network model, and/or the fourth neural network model, and the like. The storage 410 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The output interface 420 may provide processing results of the processor 130 for a user. For example, the output interface 420 may display an estimated bio-information value on a display. In this case, if the estimated blood pressure value falls outside a normal range, the output interface 420 may provide a user with warning information by changing color, line thickness, etc., or displaying an abnormal value along with a normal range, so that the user may easily recognize the abnormal value. Further, the output interface 420 may output information associated with bio-information in a non-visual manner by voice, vibrations, tactile sensation, and the like using a sound output module such as a speaker, or a haptic module and the like.

The communication interface 430 may communicate with an external device to transmit and receive various data related to estimating bio-information. The external device may include an information processing device such as a smartphone, a tablet PC, a desktop computer, a laptop computer, and the like. The communication interface 430 may communicate with the external device by using various wired or wireless communication techniques, such as Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G communications, and the like. However, this is merely exemplary and is not intended to be limiting.

In addition, the apparatus 400 for estimating bio-information may further include a trainer (not shown). The trainer may collect training data, and may train the first neural network model, the second neural network model, the third neural network model, and/or the fourth neural network model by using the collected training data. The trainer may control the pulse wave sensor 110 and the force sensor 120 to acquire pulse wave signals and force signals from a specific user or a plurality of users, and may collect the acquired signals as training data. Further, the trainer may output an interface on a display for a user to enter user characteristic information, reference blood pressure, etc., and may collect data, input by the user through the interface, as the training data. In addition, the trainer may control the communication interface 430 to receive pulse wave signals, force signals, and/or reference blood pressure values of users from an external device, such as a smartphone, a wearable device, a cuff manometer, and the like.

In this embodiment, by training a hybrid neural network model including the first neural network model, the second neural network model, and/or the fourth neural network model, which output feature vectors, and the third neural network model which estimates bio-information by using the feature vectors, and by estimating bio-information using the hybrid neural network model, accuracy of the estimation may be improved.

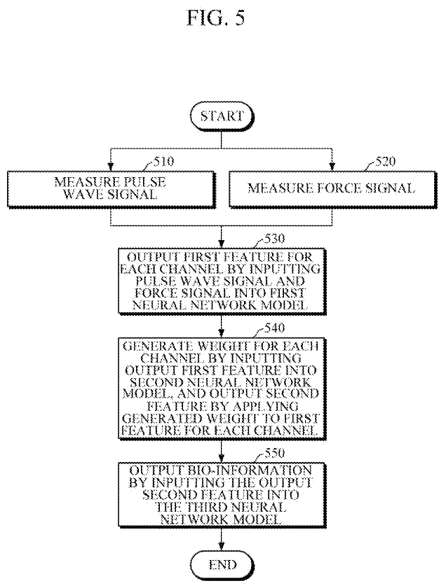

FIG. 5 is a flowchart illustrating a method of estimating bio-information according to an embodiment of the present disclosure.

The method of FIG. 5 may be an example of a method of estimating bio-information performed by the apparatuses 100 and 400 for estimating bio-information, which is described in detail above, and thus will be briefly described below.

First, when an object comes into contact with the pulse wave sensor, the apparatus for estimating bio-information may acquire a plurality of pulse wave signals for each channel from an object by using the pulse wave sensor in operation 510, and may acquire a force signal applied between the object and the pulse wave sensor by using the force sensor in operation 520.

Then, the apparatus for estimating bio-information may output a first feature for each channel by inputting the acquired force signal and pulse wave signals for each channel into the first neural network model in operation 530.

Subsequently, the apparatus for estimating bio-information may generate a weight for each channel by inputting the output first feature into the second neural network model, and may output a second feature by applying the generated weight to the first feature for each channel in operation 540.

Next, the apparatus for estimating bio-information may output bio-information by inputting the output second feature into the third neural network model in operation 550.

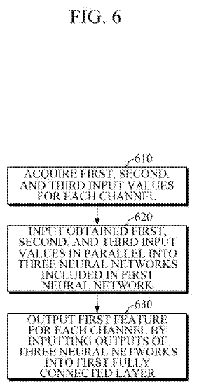

FIG. 6 is a flowchart illustrating an example of operation 530 of outputting the first feature for each channel of FIG. 5.

First, the apparatus for estimating bio-information may acquire first, second, and third input values for each channel in operation 610. The first input value may include at least one of the pulse wave signal, a first order differential signal of the pulse wave signal, and a second order differential signal of the pulse wave signal; the second input value may include at least one of an envelope of the pulse wave signal, an envelope of the first order differential signal, and an envelope of the second order differential signal which are obtained by using the force signal; and the third input value may include the force signal.

Then, the apparatus for estimating bio-information may input the obtained first, second, and third input values in parallel into three neural networks included in the first neural network in operation 620.

Subsequently, the apparatus for estimating bio-information may output the first feature for each channel by inputting outputs of the three neural networks into the first fully connected layer in operation 630.

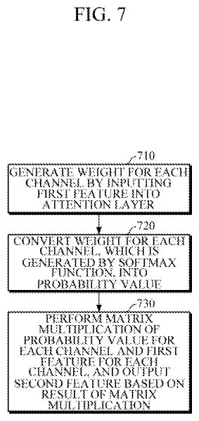

FIG. 7 is a flowchart illustrating an example of operation 540 of outputting the second feature of FIG. 5.

First, the apparatus for estimating bio-information may generate a weight for each channel by inputting the first feature into an attention layer in operation 710.

Then, the apparatus for estimating bio-information may convert the weight for each channel, which is generated by the softmax function, into a probability value in operation 720.

Subsequently, the apparatus for estimating bio-information may perform matrix multiplication of the probability value for each channel and the first feature for each channel, and may output the second feature based on results of the matrix multiplication in operation 730.

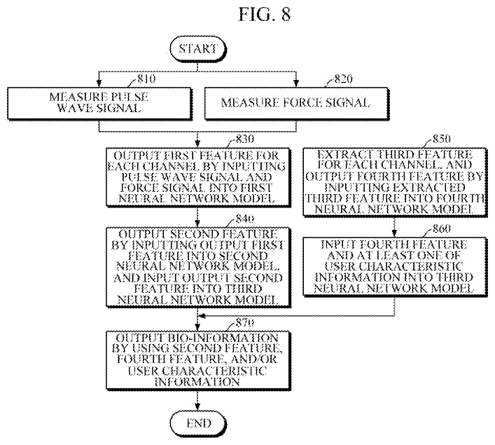

FIG. 8 is a flowchart illustrating a method of estimating bio-information in the case where a fourth neural network model is included, according to another embodiment of the present disclosure.

First, when an object comes into contact with the pulse wave sensor, the apparatus for estimating bio-information may acquire a plurality of pulse wave signals for each channel from an object by using the pulse wave sensor in operation 810, and may acquire a force signal applied between the object and the pulse wave sensor by using the force sensor in operation 820.

Then, the apparatus for estimating bio-information may output a first feature for each channel by inputting the acquired force signal and pulse wave signals for each channel into the first neural network model in operation 830.

Subsequently, the apparatus for estimating bio-information may generate a weight for each channel by inputting the output first feature into the second neural network model, may output a second feature by applying the generated weight to the first feature for each channel, and may input the output second feature into the third neural network model in operation 840.

Further, the apparatus for estimating bio-information may extract an additional third feature for each channel based on at least one of the force signal and pulse wave signals for each channel, may generate a weight for each channel based on the third feature for each channel by using the fourth neural network model, and may output a fourth feature by applying the generated weight to the third feature for each channel in operation 850.

Next, the apparatus for estimating bio-information may input the fourth feature and at least one of user characteristic information into the third neural network model in operation 860. The user characteristic information may include at least one of a user's age, stature, and weight.

Then, the apparatus for estimating bio-information may output bio-information by using the second feature, the fourth feature, and/or the user characteristic information in 870. By estimating bio-information not only based on the second feature obtained by using the second neural network model, but also based on the fourth feature, obtained by using the fourth neural network model, and/or the user characteristic information, the bio-information may be estimated more accurately.

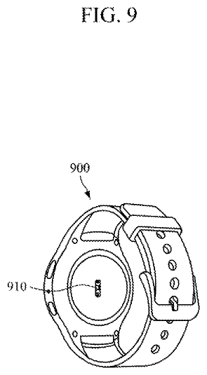

FIGS. 9 to 11 are diagrams illustrating examples of structures of an electronic device including the apparatuses 100 and 400 for estimating bio-information.

Referring to FIG. 9, the electronic device may be implemented as a wristwatch wearable device 900, and may include a main body and a wrist strap. A display is provided on a front surface of the main body, and may display various application screens, including time information, received message information, and the like. A sensor device 910 may be disposed on a rear surface of the main body to measure a pulse wave signal and a force signal for estimating bio-information.

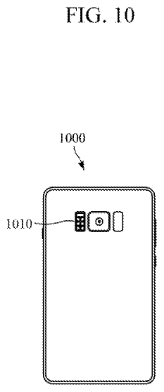

Referring to FIG. 10, the electronic device may be implemented as a mobile device 1000 such as a smartphone.

The mobile device 1000 may include a housing and a display panel. The housing may form an exterior of the mobile device 1000. The housing has a first surface, on which a display panel and a cover glass may be disposed sequentially, and the display panel may be exposed to the outside through the cover glass. A sensor device 1010, a camera module and/or an infrared sensor, and the like may be disposed on a second surface of the housing. When a user transmits a request for estimating bio-information by executing an application and the like installed in the mobile device 1000, the mobile device 1000 may estimate bio-information by using the sensor device 1010, and may provide the estimated bio-information value as images and/or sounds to the user.

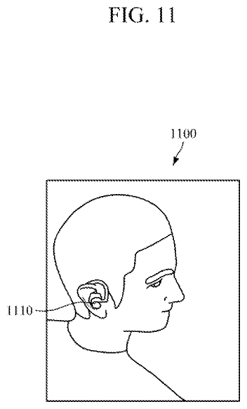

Referring to FIG. 11, the electronic device may be implemented as an ear-wearable device 1100.

The ear-wearable device 1100 may include a main body and an ear strap. A user may wear the ear-wearable device 1100 by hanging the ear strap on a user's auricle. The ear strap may be omitted depending on the type of ear-wearable device 1100. The main body may be inserted into the external auditory meatus. A sensor device 1110 may be mounted in the main body. The ear-wearable device 1100 may provide a component estimation result as sounds to a user, or may transmit the estimation result to an external device, e.g., a mobile device, a tablet PC, a personal computer, etc., through a communication module provided in the main body.

The present invention can be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the present invention can be readily deduced by programmers of ordinary skill in the art to which the invention pertains.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating bio-information, the apparatus comprising:
a pulse wave sensor having a plurality of channels to measure a plurality of pulse wave signals from an object;
a force sensor configured to obtain a force signal by measuring an external force exerted onto the pulse wave sensor; and
a processor configured to:
obtain a first feature for each channel by inputting the plurality of pulse wave signals for each channel and the force signal, into a first neural network model;
obtain a weight for each channel by inputting the first feature to a second neural network model;
obtain a second feature by applying the weight to the first feature for each channel by using the second neural network model; and
obtain bio-information by inputting the second feature to a third neural network model.

2. The apparatus of claim 1, wherein the first neural network model, the second neural network model, and the third neural network model use at least one of a Deep Neural Network, a Convolution Neural Network (CNN), and a Recurrent Neural Network (RNN).

3. The apparatus of claim 1, wherein the first neural network model comprises:
three neural networks, which are executed in parallel, and into which a first input value, a second input value, and a third input value are input respectively; and
a first fully connected layer configured to output the first feature for each channel by using outputs of the three neural networks as inputs.

4. The apparatus of claim 3, wherein the processor is further configured to:
generate a first order differential signal and a second order differential signal from the plurality of pulse wave signals, obtain at least one of the plurality of pulse wave signals, the first order differential signal, and the second order differential signal as the first input value;
generate at least one envelope, among an envelope of the plurality of pulse wave signals, an envelope of the first order differential signal, and an envelope of the second order differential signal by using the force signal;
obtain the generated at least one envelope as the second input value; and
obtain the force signal as the third input value.

5. The apparatus of claim 1, wherein the second neural network model comprises:
an attention layer configured to generate the weight for each channel by using the first feature as an input; and
a Softmax function layer configured to convert the weight for each channel into a probability value and output the probability value.

6. The apparatus of claim 5, wherein the second neural network model is configured to perform matrix multiplication of the probability value for each channel, and the first feature for each channel, and output the second feature based on results of the matrix multiplication.

7. The apparatus of claim 1, wherein the third neural network model comprises:
a second fully connected layer using the second feature as an input; and
a third fully connected layer configured to output the bio-information by using an output of the second fully connected layer as an input.

8. The apparatus of claim 1, wherein the weight for each channel based on the first feature is a first weight, and wherein the apparatus further comprises:
a fourth neural network model configured to generate a second weight for each channel based on a third feature for each channel, which is extracted based on at least one of the force signal and the plurality of pulse wave signals for each channel, and output a fourth feature by applying the weight to the third feature for each channel.

9. The apparatus of claim 8, wherein the third neural network model further comprises a fourth fully connected layer using the fourth feature and at least one of user characteristic information as an input, wherein an output of the fourth fully connected layer is input into a third fully connected layer.

10. The apparatus of claim 9, wherein the user characteristic information comprises at least one of a user's age, stature, and weight.

11. The apparatus of claim 1, wherein the bio-information comprises one or more of blood pressure, vascular age, arterial stiffness, aortic pressure waveform, vascular compliance, stress index, fatigue level, skin age, and skin elasticity.

12. A method of estimating bio-information, the method comprising:
by using a pulse wave sensor, acquiring a plurality of pulse wave signals for each channel from an object;
by using a force sensor, acquiring a force signal applied between the object and the pulse wave sensor;
obtaining a first feature for each channel by inputting the force signal and the plurality of pulse wave signals for each channel into a first neural network model;
obtaining a weight for each channel by inputting the first feature into a second neural network model;
obtaining a second feature by applying the weight to the first feature for each channel by using the second neural network model; and
obtaining bio-information by inputting the second feature into a third neural network model.

13. The method of claim 12, wherein the first neural network model, the second neural network model, and the third neural network model use on at least one of a Deep Neural Network (DNN), a Convolution Neural Network (CNN), and a Recurrent Neural Network (RNN).

14. The method of claim 12, wherein the obtaining of the first feature for each channel comprises:
obtaining a first input value, a second input value, and a third input value for each channel;
inputting the first, the second, and the third input values in parallel into three neural networks of the first neural network model; and
obtaining the first feature by inputting outputs of the three neural networks into a first fully connected layer.

15. The method of claim 14, wherein:
the first input value comprises at least one of the plurality of pulse wave signals, a first order differential signal of the pulse wave signal, and a second order differential signal of the pulse wave signal;
the second input value comprises at least one of an envelope of the plurality of pulse wave signals, an envelope of the first order differential signal, and an envelope of the second order differential signal which are generated by using the force signal; and
the third input value comprises the force signal.

16. The method of claim 12, wherein the obtaining of the second feature comprises:
generating the weight for each channel by inputting the first feature into an attention layer; and
converting the weight for each channel into a probability value by using a Softmax function.

17. The method of claim 16, wherein the obtaining of the second feature further comprises obtaining the second feature by performing matrix multiplication of the probability value for each channel and the first feature for each channel.

18. The method of claim 12, wherein the obtaining the bio-information comprises:
inputting the second feature into a second fully connected layer; and
obtaining the bio-information by inputting an output of the second fully connected layer into a third fully connected layer.

19. The method of claim 12, wherein the weight for each channel based on the first feature is a first weight, and the method further comprises:
generating a second weight for each channel based on a third feature for each channel, which is extracted based on at least one of the force signal and the plurality of pulse wave signals for each channel, by using a fourth neural network model; and
obtaining a fourth feature by applying the second weight to the third feature for each channel.

20. The method of claim 19, wherein the obtaining the bio-information comprises:
inputting the fourth feature and at least one of user characteristic information into a fourth fully connected layer; and
outputting the bio-information by inputting an output of the fourth fully connected layer into a third fully connected layer.
